(19) United States
(12) Patent Application Publication
(10) Pub. No.: US 2021/0134441 A1
(43) Pub. Date: May 6, 2021

(54) SELECTION AND PERFORMANCE OF HOSTED AND DISTRIBUTED IMAGING ANALYSIS SERVICES

(71) Applicant: Daniel Marcus, St. Louis, MO (US)

(72) Inventor: Daniel Marcus, St. Louis, MO (US)

(73) Assignee: Washington University, St. Louis, MO (US)

(21) Appl. No.: 17/088,983

(22) Filed: Nov. 4, 2020

Related U.S. Application Data
(63) Continuation of application No. 12/634,392, filed on Dec. 9, 2009, now abandoned.
(60) Provisional application No. 61/121,068, filed on Dec. 9, 2008.

(51) Int. Cl. G16H 30/40

(52) U.S. Cl. CPC G16H 30/40; G16H 50/20

(57) ABSTRACT

A patient's image data and associated metadata describing the image data and characteristics of the patient are received and stored in a memory area. Information that describes one or more imaging analysis services and associated service criteria is accessed and compared to the metadata. Based on matches between the image metadata and service criteria, services are selected and executed, producing an output. Iterative rounds of service matching based on the metadata and the output of previous services and service execution proceed. The output of the services is associated with the image data and metadata, stored in the memory area, and made available to the user. A data repository, including the original images, metadata, and service output is accessible via the services for use in comparative analyses. Such services can utilize the repository to dynamically update indices and algorithms used in the comparative analyses.

### CROSS REFERENCE TO RELATED APPLICATION

This application is a continuation of U.S. application Ser. No. 12/634,392 filed on Dec. 9, 2009, the disclosure of which is hereby incorporated by reference in its entirety. U.S. application Ser. No. 12/634,392 claims the benefit of U.S. Provisional Application No. 61/121,068, filed 9 Dec. 2008, the disclosure of which is hereby incorporated by reference in its entirety.

### STATEMENT REGARDING FEDERALLY SPONSORED RESEARCH & DEVELOPMENT

This invention was made with government support under grants P30 NS048056 and U24 RR021382 awarded by the U.S. National Institutes of Health. The government has certain rights in the invention.

### BACKGROUND

Present state-of-the-art medical imaging information systems store, process, and distribute medical scans to facilitate physicians in diagnosing and treating disease. These systems are generally closed systems requiring the use of specific hardware along with proprietary software for their use.

These present systems generally require localized storage of patient data, images and analysis results, incorporate localized data processing systems, and do not provide an open or standardized framework to enable third-parties to expand upon the capabilities of such systems. As such, the capabilities of the present systems are often restricted to the abilities of the vendor of such systems. These systems also do not provide mechanisms to detect image characteristics that would guide selection and application of potentially informative image analysis methods.

These restrictions limit the ability of physicians and other consumers of medical images to access and utilize quantitative software-based image analysis methods. The use of such quantitative methods is emerging as an important advance in patient care, personalized medicine, biomedical research, and development of drugs, devices and other interventions. A system that enables the efficient delivery of such services is therefore highly desirable.

### SUMMARY

Embodiments of the invention describe a method of networked imaging analysis. A patient's image data and associated metadata are received. The metadata describes the image data and characteristics of the patient. Such characteristics could include, but are not limited to, clinical history and demographic information. The image data and metadata are stored in a memory area. Information that describes one or more imaging analysis services and associated service criteria, and which is stored in the memory area, is accessed and compared to the metadata. Based at least on matches between the image metadata and service criteria, services are selected and executed, producing an output. Iterative rounds of service matching and service execution proceed. Service matching in these iterative rounds is based on metadata and/or the output of previous services. The output of the services is associated with the image data and metadata, stored in the memory area, and made available to the user. A data repository, including the original images, metadata, and service output is accessible via the services for use in comparative analyses. Such services can utilize the repository to dynamically update indices and algorithms used in the comparative analyses.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is an exemplary block diagram illustrating a medical imaging analysis system.

FIG. 2 is an exemplary block diagram illustrating a hosting computing device.

FIG. 3 is an exemplary flow chart illustrating the computing of certain imaging analysis services.

FIG. 4 is an exemplary flow chart illustrating the process by which a user selects imaging analysis services to be displayed.

FIG. 5 illustrates an exemplary implementation of an imaging analysis system for storing patient data and service output data such that access latency and storage efficiency are balanced by the likelihood that such data will be accessed by user.

Corresponding reference characters indicate corresponding parts throughout the drawing

### DETAILED DESCRIPTION

Referring to the figures, embodiments of the invention host one or more imaging analysis services 108. Exemplary imaging analysis services 108 include computational analysis of image data 112 from scans such as computerized axial tomography (CAT) scans, magnetic resonance imaging (MRI) scans, or positron emission tomography (PET) scans. The services provide valuable insight to clinicians, patients, and other users 102 regarding the diagnosis and treatment of various diseases, conditions, and anatomical and physiological states. Aspects of the invention enable a computing device to host a plurality of such services, providing user 102 with a single source for accessing the services. In some embodiments, the services are performed remotely, such as by “cloud computing.” Cloud computing is an abstraction concept in which computations are distributed among a plurality of networked computing devices for simultaneous executions, thus speeding the performance of the computations. In some embodiments, the executables to perform the services are dynamically discovered in remote libraries, dynamically installed onto a local environment, and executed on that local environment.

Embodiments of the invention enable the filtering, sorting, pre-selection, and execution of the imaging analysis services 108 based on the incoming image data 112 and image metadata 114. The filtered services are indicated to user 102 in a user interface. In further embodiments, one or more of the filtered services are performed without an explicit request from user 102, as described in detail below. The pre-computing aspect of the invention improves the user experience by reducing the amount of time between receiving a request from user 102 and providing the results of the analysis. In some embodiments, the response is in real-time or near real-time. In alternative embodiments, additional imaging analysis services 108 are triggered and performed based on the output of one or more imaging analysis services 108 in an iterative process.

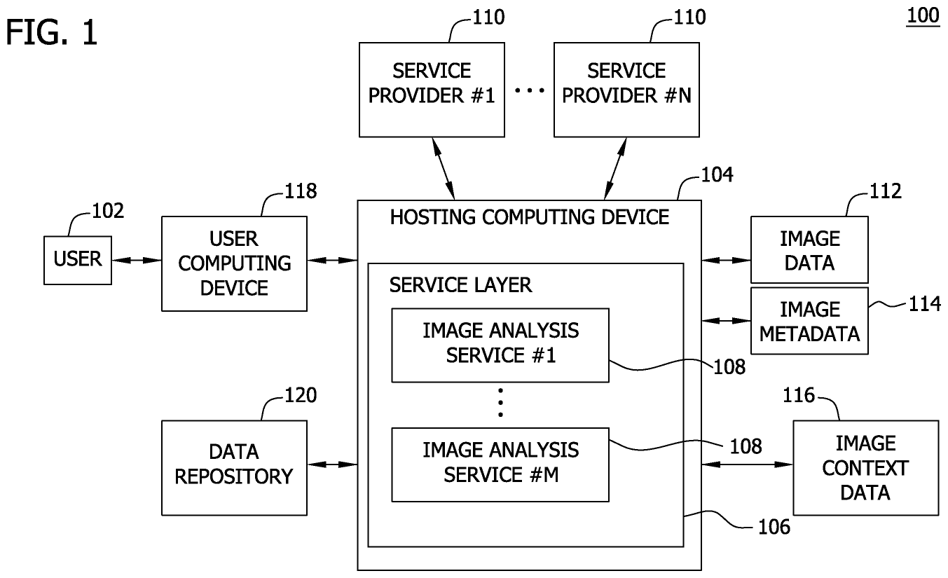

Referring to FIG. 1, an exemplary block diagram illustrates a medical imaging analysis system 100 for use by user 102. In an exemplary embodiment, a hosting computing device 104 includes a service layer 106 for storing data describing one or more imaging analysis services 108 as well as hosting execution of imaging analysis services 108. Data describing one or more imaging analysis services 108 may be provided by one or more third-party service providers 110, or may be pre-defined by system 100. The hosting computing device 104 receives image data 112 and image metadata 114 associated with a patient (not shown), wherein image metadata 114 describes image data 112, one or more characteristics of the patient, and one or more characteristics of an imaging context 116. Imaging context characteristics 116 may include user preferences and instructions. Imaging context characteristics may also include a standard analysis protocol to be executed for a particular research study or clinical trial. In various embodiments, image data 112 and image metadata 114 are sent to medical imaging analysis system 100 by one or more of image acquisition devices directly, hardware and/or software processes relaying data from image acquisition devices to system, medical imaging information systems including Picture Archiving and Communication Systems (PACS), image workstations such as those used commonly by physicians to access and visualize medical images, user controlled upload to system 100, and importing directly from recordable media into system.

Hosting computing device 104 accesses data describing imaging analysis services 108 and metadata 114 describing one or more characteristics of the patient. Hosting computing device 104 compares the metadata 114 to data describing imaging analysis services 108, selects one or more of imaging analysis services 108 based on the comparison, and identifies the selected imaging analysis services 108 to user 102 through user computing device 118. An initial round of services may be executed, based on matched criteria, and the results of these services are used to further identify matching services. Such matching and service execution may continue in an iterative fashion. Hosting computing device 104 then receives a request from user 102 to execute one or more of imaging analysis services 108. In response to such request, hosting computing device 104 manages execution of imaging analysis services 108 based on image data 112 and associated metadata 114 to generate an output (not shown). Further, hosting computing device 104 stores the output for later retrieval and display to user 102 through user computing device 118. In some embodiments, the term “patient” includes any biological organism or combination thereof, such as a human, animal, or any portion of such that is capable of being imaged using a medical imaging device, and may include those imaged for diagnostic, therapeutic, research, educational, informational, and other purposes.

A persistent data repository 120 is generated and includes image data 112 and metadata 114 submitted to system 100 and the output from services executed on image data 112 and metadata 114. In some embodiments, imaging analysis services 108 utilize the contents of data repository 120 to make inferences about image data 112 such as newly acquired image data 112 or previously stored image data 112. Imaging analysis services 108 may dynamically update its operators based on the current contents of data repository 120. For example, in one embodiment, imaging analysis service 108 retrieves total brain volumes for similar patients, such as based on one or more of age and gender, and determines whether a patient's total brain volume is abnormal. Service criteria 208 may also utilize the data repository to define whether image data 112 match a particular service 108.

In an exemplary embodiment, user computing device 118 displays output using graphics and text displayed on a computer monitor, wherein the graphics are one or more of two-dimensional image slices of data and three-dimensional renderings of images. In an alternative embodiment, user computing device 118 displays output using standardized printable electronic document formats such as portable document format or Digital Imaging and Communications in Medicine (DICOM) structured reports. In another alternative embodiment, the output is sent to user computing device 118, for instance, when user computing device 118 is a medical information system such as a PACS. In multiple alternative embodiments, medical imaging analysis system 100 provides for interface views for multiple types of users 102, including a general consumer, medical practitioner, and clinical trial user. In an exemplary embodiment, the general consumer user interface view provides user 102 access to the patient/user's image data 112, metadata 114, and output from imaging analysis services 108. The information is presented in a manner that is understandable to non-experts, and includes one or more of anatomic annotations, pathologic annotations, and links to reference information and normative data retrieved from data repository 120 and other data sources. In various alternative embodiments, targeted links to one or more of relevant therapies, products, clinical trials, support groups, and other information are provided to the general consumer user based on the relevancy to the displayed output. In another alternative embodiment, social networking type tools enable the general consumer user to communicate with other general consumer users who have similar or related outputs from imaging analysis services 108. In another alternative embodiment, the information is embedded in a third party personal health record system.

In an alternative embodiment, output is displayed for a medical practitioner, and the medical practitioner interface view supports a standard workflow implemented in radiology clinics. In the medical practitioner interface view, one or more of image data 112, metadata 114, and output are accessible, as well as links to relevant imaging analysis services 108 available to the medical practitioner to select, including information as to why such imaging analysis service 108 is included in the links. In an alternative embodiment, links are provided to relevant imaging analysis services 108 provided by third-party service providers 110. In an exemplary embodiment, using the medical practitioner interface view, the output of imaging analysis services 108 are displayed in reports suitable for distribution to patients and referring physicians. In another alternative embodiment, output is displayed for a clinical trial user in a clinical trial interface view that emphasizes standardized analytic workflows and provides access to data by clinical trial architecture. Non-imaging data related to a clinical trial, such as drug dose or diagnosis, are selected and displayed alongside the patient's image data 112 and output of imaging analysis services 108. In another alternative embodiment, external applications (not shown) programmatically retrieve the output via an Application Programming Interface (API) for display.

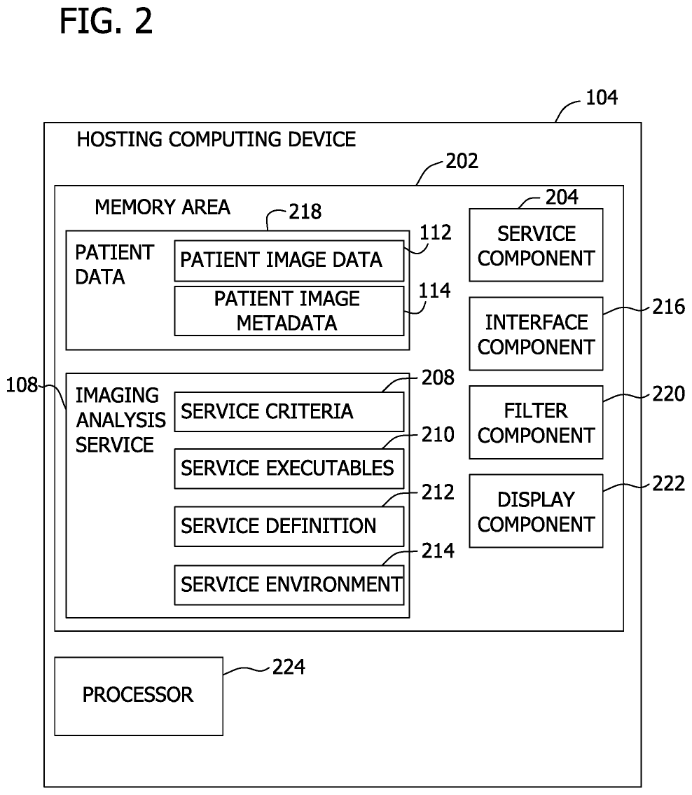

FIG. 2 is an exemplary block diagram illustrating the hosting computing device 104 such as is provided for in FIG. 1. Hosting computing device 104 includes a memory area 202 having a plurality of computer-executable components. Memory area 202 of hosting computing device 104 includes a service component 204 which provides for hosting one or more imaging analysis services 108 including one or more of a service criteria 208, service executables 210, service definition 212, and service environment 214 associated with imaging analysis service 108. Memory area 202 also includes an interface component 216 for receiving patient data 218 such as image data 112, metadata 114 describing image data 112 and one or more patient characteristics and image context data 116. Further, memory area 202 includes a filter component 220 for selecting only imaging analysis services 108 having associated service criteria 208 corresponding to one or more of metadata 114 and output of previously executed imaging analysis services 108. Memory area 202 includes a display component 222 for identifying imaging analysis services 108 to user 102 (not shown in FIG. 2). In one alternative embodiment, filtering component 220 filters the retrieved services. In another alternative embodiment, one or more of filtered and unfiltered services are retrieved programmatically by external applications using interface component 216 and subsequently displayed to user 102 via the external application.

In an exemplary embodiment, user 102 interacts with display component 222 to manage execution of at least one imaging analysis service 108. In an alternative embodiment, user 102 manages the execution of at least one imaging analysis service 108 by interacting with external applications that communicate with hosting computing device 104 through interface component 216. In the exemplary embodiment, service criteria 208 describes image data 112, image metadata 114, and image context values or range of values that are used to match imaging analysis service 108 to image data 112. Further, in the exemplary embodiment, service executables 210 include computer instructions for use in executing imaging analysis service 108, service definition 212 includes a series of conditional steps and input parameters for service executables 210 for use in executing imaging analysis service 108, and service environment 214 includes a description of requirements for a computing system to execute imaging analysis service 108. The requirements include operating system type, memory requirements, and other hardware and software details. In an alternative embodiment, service environment 214 includes a virtualized computing environment preconfigured with the requirements to execute imaging analysis service 108, and components of imaging analysis service 108 are preconfigured on hosting computing device 104. In another alternative embodiment, the components of imaging analysis service 108 are received by hosting computing device 104 through interface component 216. In an exemplary embodiment, service execution component 204 manages the execution of imaging analysis services 108 requested by user 102 and imaging analysis services 108 that are automatically executed. Further, in the exemplary embodiment, service execution component 204 deploys services executables 210 on a local processor 224, launches service executables 210 in the sequence described in service definition 212 and using the input parameters described in service definition 212, monitors the operation of imaging analysis service 108, and provides status notifications to user 102 via display component 222 and interface component 216. In an alternative embodiment, processor 224 is a network accessible processor upon which executables can be deployed. In yet another embodiment, processor 224 is a network accessible processor upon which a virtualized operating system and associated executables can be deployed.

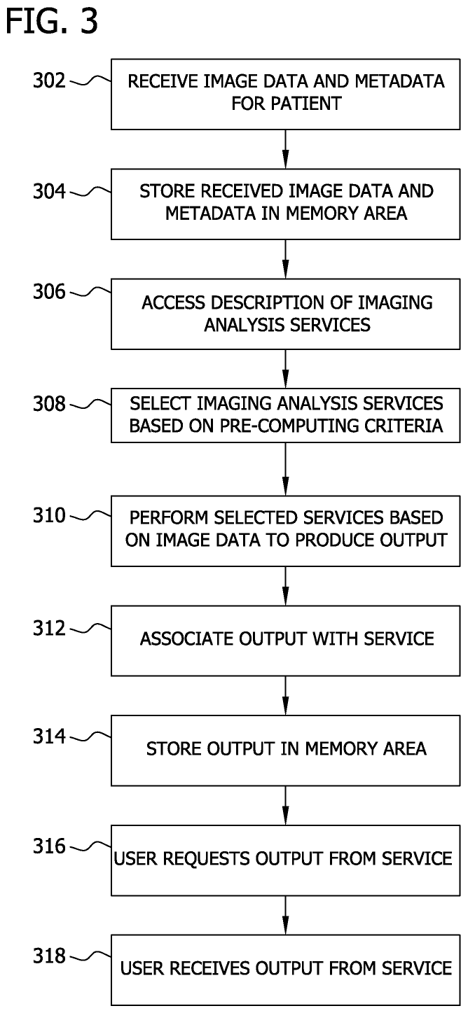

FIG. 3 is an exemplary flow chart illustrating the method of computing certain imaging analysis services 108 by one or more of hosting computing devices 104 and a medical imaging analysis system (not shown in FIG. 3) such as system 100. In various embodiments, image data 112, image metadata 114, and image context data 116 are sent to medical imaging analysis system 100 by one or more of image acquisition devices directly, hardware and/or software processes relaying data from image acquisition devices to system 100, user controlled upload to system 100, and importing directly from recordable media into system 100. The method includes receiving at 302 patient image data 112, metadata 114, and image context data 116, wherein metadata 114 describes the image and one or more characteristics of the patient, and context data 116 describes directives to the system. The method also includes storing at 304 the patient image data 112 and metadata 114, including other patient characteristics, in memory area 202 (not shown in FIG. 3), accessing at 306 data describing the imaging analysis services 108 stored in memory area 202, selecting at 308 one or more of the imaging analysis services 108 based on a pre-computing criteria, and performing at 310 each of the selected imaging analysis services 108 automatically based on image data 112 and/or the metadata 114, including other patient characteristics, to produce a corresponding output. Further, the produced output is associated at 312 with the corresponding imaging analysis service 108, and the output is stored at 314 in memory area 202. Subsequently, user 102 requests at 316 the output corresponding to at least one of the selected imaging analysis services 108, and user 102 receives at 318 the output. Associating produced output with one or more of the corresponding imaging analysis services 108, image data 112, and image metadata 114, enables system 100 to retrieve the produced output, image data 112, and image metadata 114 of a particular imaging analysis service 108 based upon a user's request 316 for such output. Alternatively, the output can be pushed to the user according to directives supplied in image context data 116.

In an exemplary embodiment, user 102 requests at 316 output corresponding to imaging analysis services 108 wherein imaging analysis services 108 have already been performed and thus user 102 receives at 318 the output almost immediately. In an alternative embodiment, user 102 requests at 316 output corresponding to imaging analysis services 108 that have not yet been performed, thus the medical imaging analysis system is directed to perform the selected services based on image data 112, metadata 114, and/or context data 116 to produce the output, and only then does user 102 receive at 318 the output. In an exemplary embodiment, the pre-computing criteria used to select at 308 imaging analysis services 108 is the frequency of execution of each of the selected imaging analysis services 108. In alternative embodiments, the pre-computing criteria includes one or more of a cost of executing each of the selected imaging analysis services 108, a relevancy of each of the selected imaging analysis services 108 to image data 112, and a relevancy of each of the selected imaging analysis services 108 to the metadata 114. In an alternative embodiment, pre-computing criteria include comparisons of patient's image data 112 and metadata 114 with other patients' image data 112 and metadata 114 as well as other patients' output from imaging analysis services 108. In another alternative embodiment, imaging analysis services 108 are selected based on a comparison of the service criteria to the produced output of other previously executed imaging analysis services. Similarly, in another alternative embodiment, imaging analysis services 108 includes a service for comparing patient's image data 112 and metadata 114 against the patient's past accumulated image data 112 and metadata 114 to determine if any anatomic and functional changes may indicate potential disease.

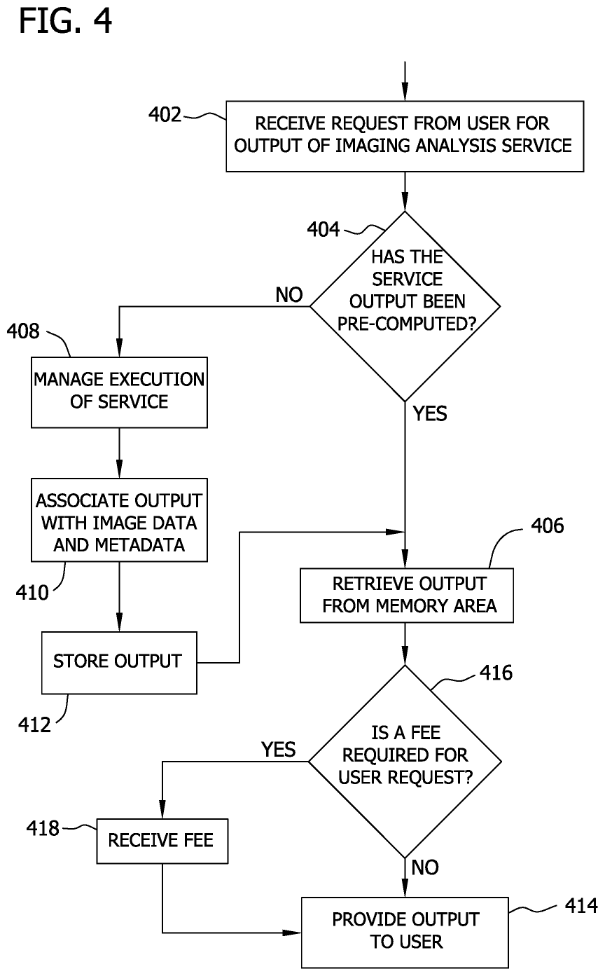

FIG. 4 is an exemplary flow chart illustrating the process by which one or more of hosting computing devices 104 (not shown in FIG. 4) and a medical imaging analysis system (not shown in FIG. 4) such as medical imaging analysis system 100 communicate with user 102 (not shown in FIG. 4) selecting imaging analysis services 108 (not shown in FIG. 4) to be provided to user 102. A request is received at 402 from user 102 for output of imaging analysis service 108 (not shown in FIG. 4). A determination at 404 is made whether the output has been pre-computed and can be retrieved at 406 from memory area 202 immediately. If not, the execution of the service is managed at 408, the output is associated at 410 with patient image data 112 and metadata 114 (not shown in FIG. 4), and the output is stored at 412 in memory area 202. The output is then retrieved at 406 from memory area 202. Prior to providing output at 414 to user 102, a determination at 416 is made as to whether a fee is applicable for the user's request. If so, the fee is requested and received at 418, and the output is provided at 414 to user 102. In an exemplary embodiment, the fee is received via user input through user computing device 118 (not shown in FIG. 4) authorizing the system to deduct the fee from a credit account. In alternative embodiments, the fee is received at 418 via any suitable payment method known to those skilled in the art and guided by the teachings herein provided that are capable of performing the functions as described herein.

In an exemplary embodiment, the imaging analysis services 108 for which requests are received at 402 from user 102 more frequently are pre-computed and available to be retrieved at 406 almost immediately. In an alternative embodiment, imaging analysis services 108 are pre-computed based on criteria from one or more third-party service providers 110 (not shown in FIG. 4). In another alternative embodiment, imaging analysis services 108 are pre-computed based on directives in image context data 116. In other alternative embodiments, imaging analysis services 108 are pre-computed based on one or more of the following criteria: a cost of executing each of the selected imaging analysis services 108, a relevancy of each of the selected imaging analysis services 108 to image data 112, and a relevancy of each of the selected imaging analysis services 108 to metadata 114. In an alternative embodiment, all of imaging analysis services 108 for which a request is received at 402 from user 102 have been pre-computed and are available to be retrieved at 406 from memory area 202 almost immediately.

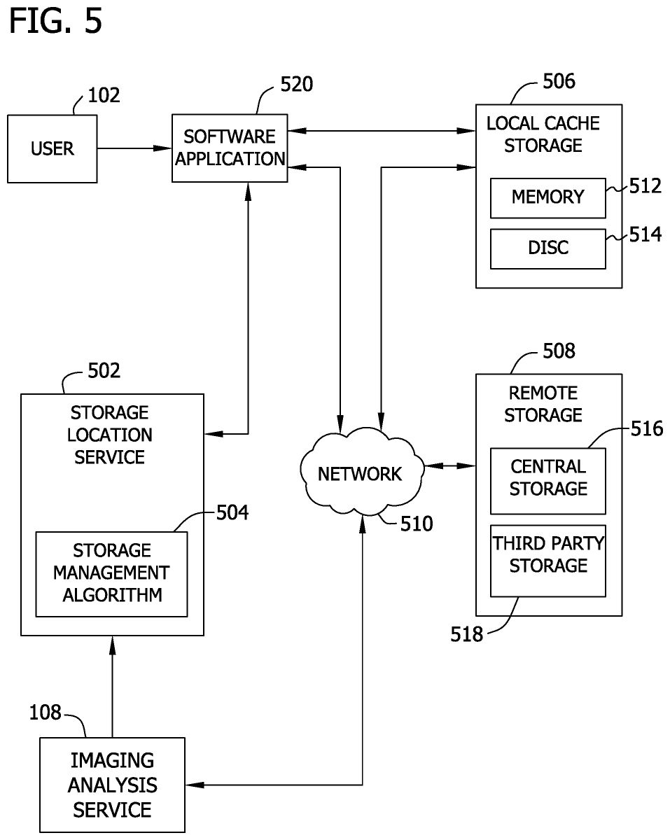

FIG. 5 illustrates an exemplary implementation of an imaging analysis system 500 for storing patient data 218 and service output data such that access latency and storage efficiency are balanced by the likelihood that such data will be accessed by user 102. Upon execution of imaging analysis service 108, a storage location service 502 that includes a storage management algorithm 504 directs the storage of data in a storage location. In an exemplary embodiment, storage management algorithm 504 is a high likelihood algorithm used to determine what resources are stored in a local cache storage 506 and what resources are stored in remote storage 508 accessible via a network 510. Further, when data are to be stored in local cache storage 506, storage management algorithm 504 determines within which tier of cache data are stored, including one or more of a local memory 512 and a local disk 514. In an exemplary embodiment, algorithm 504 determines the storage location of resources based on the likelihood of immediate access of data based on the workflow of user 102. For example, in an exemplary embodiment, patient data 218 and service output data scheduled to be viewed by a medical professional are stored in local cache storage 506 on local disk 514, along with prior patient data 218 and service output data for one or more patients in a viewing queue; and patient data 218 along with service output data for a next patient is stored in local cache storage 506 in the faster local memory 512. Similarly, patient data 218 for patients not in the viewing queue is stored on one or more of a remote central storage 516 and remote third-party storage 518. In various embodiments, communication over network 510 uses one or more network protocols, such as HTTP, FTP, SSH, and DICOM. In an exemplary embodiment, remote central storage 516 is a data repository located on high performance remote hardware. User 102 requests data via software application 520 which communicates with storage location service 502 to determine the location of the requested data. Software application 520 retrieves the data from the identified location and makes it available to user 102. In an alternative embodiment, output data are sent to a PACS and not managed by storage location service 502.

Exemplary Operating Environment

A computer or computing device such as described herein has one or more processors or processing units, system memory, and some form of computer readable media. By way of example and not limitation, computer readable media comprise computer storage media and communication media. Computer storage media include volatile and nonvolatile, removable and non-removable media implemented in any method or technology for storage of information such as computer readable instructions, data structures, program modules or other data. Communication media typically embody computer readable instructions, data structures, program modules, or other data in a modulated data signal such as a carrier wave or other transport mechanism and include any information delivery media. Combinations of any of the above are also included within the scope of computer readable media.

The computer may operate in a networked environment using logical connections to one or more remote computers, such as a remote computer. Although described in connection with an exemplary computing system environment, embodiments of the invention are operational with numerous other general purpose or special purpose computing system environments or configurations. The computing system environment is not intended to suggest any limitation as to the scope of use or functionality of any aspect of the invention. Moreover, the computing system environment should not be interpreted as having any dependency or requirement relating to any one or combination of components illustrated in the exemplary operating environment. Examples of well known computing systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Embodiments of the invention may be described in the general context of computer-executable instructions, such as program modules, executed by one or more computers or other devices. The computer-executable instructions may be organized into one or more computer-executable components or modules. Generally, program modules include, but are not limited to, routines, programs, objects, components, and data structures that perform particular tasks or implement particular abstract data types. Aspects of the invention may be implemented with any number and organization of such components or modules. For example, aspects of the invention are not limited to the specific computer-executable instructions or the specific components or modules illustrated in the figures and described herein. Other embodiments of the invention may include different computer-executable instructions or components having more or less functionality than illustrated and described herein. Aspects of the invention may also be practiced in distributed computing environments where tasks are performed by remote processing devices that are linked through a communications network. In a distributed computing environment, program modules may be located in both local and remote computer storage media including memory storage devices.

The embodiments illustrated and described herein as well as embodiments not specifically described herein but within the scope of aspects of the invention constitute exemplary means for hosting the imaging analysis services 108 on the system, exemplary means for selecting relevant imaging analysis services 108 based on image data 112 and associated metadata 114, and exemplary means for performing one or more of the imaging analysis services 108 upon receipt of image data 112 without instruction from user 102.

The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

When introducing elements of aspects of the invention or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense

What is claimed is:

1. A system for medical imaging analysis, said system comprising:
a memory area for storing data describing one or more imaging analysis services, said data including service criteria associated with the imaging analysis services; and
a processor programmed to:
receive image data and metadata associated therewith for a patient, said metadata describing the image data and one or more characteristics of the patient;
access the data describing the imaging analysis services and associated service criteria stored in the memory area;
compare the received metadata to the accessed service criteria;
select one or more of the imaging analysis services based on said comparison; and
identify the selected imaging analysis services to a user.

2. The system of claim 1, wherein said processor is further programmed to:
receive a request from the user to execute one or more of the selected imaging analysis services;
responsive to the request, manage execution of the one or more of the selected imaging analysis services based on the received image data and associated metadata to produce an output;
associate the produced output with the received image data and metadata; and
store said output in said memory area.

3. The system of claim 1, wherein said processor is further programmed to:
determine a subset of the selected imaging analysis services based on pre-computing criteria prior to identifying the selected imaging analysis services to the user;
manage execution of said determined subset of the selected imaging analysis services to produce an output;
associate the produced output with the received image data and associated metadata; and
store said output in said memory area.

4. The system of claim 3, wherein the processor is programmed to determine the subset based on the pre-computing criteria comprising one or more of the following: a cost of executing each of the selected imaging analysis services, a relevancy of each of the selected imaging analysis services to the image data, a relevancy of each of the selected imaging analysis services to the metadata, and a frequency of execution of each of the selected imaging analysis services.

5. The system of claim 3, wherein said processor is further programmed to:
receive a request for the output;
retrieve the output from said memory area; and
provide the retrieved output to the user.

6. The system of claim 3, wherein said processor is further programmed to:
retrieve one or more parameters from a persistent data repository prior to execution of each imaging analysis service;
dynamically update the imaging analysis services using the retrieved parameters prior to execution of the imaging analysis services; and
store the image data, associated metadata, and the produced output, after execution of the imaging analysis services, in the persistent data repository;

7. The system of claim 1, wherein the processor is programmed to charge a fee to the user for requesting the execution of one or more imaging analysis services.

8. The system of claim 1, further comprising:
means for hosting the imaging analysis services on the system;
means for selecting relevant imaging analysis services based on the image data and associated metadata; and
means for performing one or more of the imaging analysis services upon receipt of the image data without instruction from the user.

9. A method comprising:
receiving image data and metadata associated therewith for a patient, said metadata describing the image data and one or more characteristics of the patient;
storing the image data and metadata in a memory area;
accessing data describing one or more imaging analysis services stored in the memory area;
selecting one or more of the imaging analysis services based on pre-computing criteria;
performing each of the selected imaging analysis services based on the received image data to product a corresponding output;
associating the produced output with the corresponding image analysis service; and
storing the associated, produced output in the memory area, wherein a user subsequently requests and receives the output corresponding to at least one of the selected imaging analysis services.

10. The method of claim 9, wherein the accessed data includes service criteria describing the imaging analysis services, and wherein selecting the one or more imaging analysis services comprises selecting the one or more imaging analysis services based on a comparison of the service criteria with the metadata.

11. The method of claim 9, further comprising providing the output for display to the user.

12. The method of claim 9, further comprising assessing a fee to the user for requesting the output of corresponding to at least one of the selected imaging analysis services.

13. The method of claim 9, further comprising selecting one or more of the imaging analysis services based on a comparison of the service criteria to the produced output of imaging analysis services.

14. One or more computer-readable media having computer-executable components, said components comprising:
a service component for hosting one or more imaging analysis services, said service component storing one or more of data describing each of the imaging analysis services, service criteria associated therewith, and software components for executing said service;
an interface component for receiving image data and metadata associated therewith for a patient, said metadata describing the image data and one or more characteristics of the patient;
a filter component for selecting only the imaging analysis services having the associated service criteria correspond to the metadata; and
a display component for identifying the imaging analysis services selected by the filter component to a user, wherein the user interacts with the interface component to manage execution of one or more of the identified imaging analysis services based at least on the image data.

15. The computer-readable media of claim 14, wherein the service component further defines one or more of the imaging analysis services, associated criteria, and software components for executing said service.

16. The computer-readable media of claim 14, wherein the interface component further receives the data describing the imaging analysis services and associated service criteria.

17. The computer-readable media of claim 14, wherein the service component further manages execution of one or more of the imaging analysis services based at least on the image data to produce an output and associates the produced output with the image data and metadata.

18. The computer-readable media of claim 17, wherein the interface component further receives a request from the user to execute one or more of the imaging analysis services, and wherein the service component further provides to the user the produced output associated with the image data.

19. The computer-readable media of claim 17, wherein the imaging analysis services are hosted by a service provider, and wherein the service component manages execution of the one or more of the imaging analysis services by requesting execution of the imaging analysis services by the service provider, wherein the service provider produces the output, and wherein the interface component further receives the produced output from the service provider and provides the produced output to the user.

20. The computer-readable media of claim 19, wherein the display component provides identification of the imaging analysis services selected by the filter component to a computing device associated with the user, and wherein the computing device displays the identified imaging analysis services to the user.
